(19) United States
(12) Patent Application Publication
(10) Pub. No.: US 2021/0021423 A1
(43) Pub. Date: Jan. 21, 2021

(54) PROCEDURE OF UNIFIED GLOBAL REGISTRATION AND UNIVERSAL IDENTIFICATION OF SPATIALLY LOCATABLE OBJECTS

(71) Applicant: CONÉCTATE SOLUCIONES Y APLICACIONES SL, Soria (ES)

(72) Inventors: Fernando Latorre, Soria (ES); Nuria Sala Cano, Soria (ES)

(21) Appl. No.: 16/744,314

(22) Filed: Jan. 16, 2020

(30) Foreign Application Priority Data
Jul. 20, 2019 (ES) 201931249U

(51) Int. Cl. H04L 9/32; G06Q 10/10; G06Q 30/00; G16H 10/60; G06Q 50/26; H04L 29/06

(52) U.S. Cl. CPC H04L 9/3236; G06Q 10/10; G06Q 30/018; G16H 10/60; H04W 4/80; H04L 63/101; G06Q 2220/00; H04L 2209/38; G06Q 50/265

(57) ABSTRACT

A procedure of unified registration and universal identification in any territory of spatially locatable objects, in order to achieve interoperability between objects or spatial locations, different types of computing systems, the procedure comprising: generation of a unique and non-transferable identifier for each connected, smart and spatial object device, in the URN UUID, OID and DID formats, without discarding others; creation of a DID document associated with the generated identifier; association of different identifiers of the object and of the user/owner; assignment of permissions, roles and creation of access control list (ACL); creation of encryption keys associated with the ACL; save in the DID document both data, service identifiers and other associated identifiers, as well as necessary permissions for accessing to object data; digital fingerprint generation of the DID document using hash; associated data and hash storage in the system and in one or more DLTs or blockchain networks.

### TECHNICAL FIELD

The field of application is part of the sector of the identification and traceability of devices, geographical and spatial systems, while covering healthcare, electronics, robotics, communications, biotechnology, nanotechnology and data management systems.

### BACKGROUND OF THE INVENTION

Spatial object is the digital representation of geographical entity or phenomenon, which forms the basis for data management and analysis; a good classification model should not only be good for representing real entities, but also good for revealing spatial relationships, therefore good for formalizing spatial analyses.

IoT (Internet of Things) can be developed in many areas and in environments where countless devices of different nature and technical profile will operate, manufactured in turn by thousands of different brands (each with their own standards). The market for IoT wireless connectivity is therefore highly fragmented. Options include ANT+, Bluetooth, Bluetooth Smart, EnOcean, InGenu, LoRa, LTE, NB-IoT (essentially the subset of 5G IoT use cases), Sigfox, Sub-GHz, ULE Alliance, Weightless, WiGig and various types of WiFi. Developing the ability for all of them to communicate with each other is not only a technical challenge, but also a matter of uniformity. That's why IoT interoperability comes as a necessity.

In the case of robots, these are virtual entities or artificial physical devices, capable of performing a variety of tasks, based on instructions programmed in advance or sent remotely. There are different types of robots, e.g. industrial, domestic or medical robots (such as surgery robots, some machines to lift people, . . . ), entertainment robots (toy robots), terrestrial, air or submarine robots (vehicles and other robots), humanoids (looking human-like and performing human-specific tasks). In addition, an autonomous vehicle, also known as robotic, smart, driverless or self-driving, is capable of mimicking human driving and control capabilities, as well as perceiving the environment around it and operating accordingly. These devices feed off information from smart sensors, maps and calculations, based on algorithms, and perceive the environment using complex techniques.

Mobile devices such as electronic devices and vehicles must communicate with others to allow mutual identification, location and coordination of their movements, carried out in such a way that certain public information of the device and the privacy of the private information associated with that device can also be guaranteed. Identifying connected and smart devices is therefore an important issue in the Internet of Things (IoT) and plays a relevant role. Unification in identification promotes interoperability and is essential for the proper development of IoT and robotics. However, there is currently no known system for doing this.

There is also no unified identification of robots. Only OWASP (Open Web Application Security Project) has proposed a project called “Robot Security Project” to perform a robot identification, but which is undeveloped, and which suggests that industry and administrations use systems for registering identifiers that mitigate the risk that someone might manipulate them. Currently, there is no unified system for identifying connected and smart objects and devices, but only manufacturer-specific identification systems and some standards for different uses. For example, a robot has to generate its own universal identifier by itself, forcing it to incorporate the logic or electronics needed to internally generate that identifier (for example, with a software algorithm for generating UUIDs or with a reverse-polarized diode as a hardware random number generator) or use an external service to be generated, and thus obtain the internal identifier to add it to the device's own internal memory. These mechanisms, however, do not ensure that there cannot be a collision with another (possibility of two identical identifiers being generated), nor that that identifier can be recognized by other devices, in the absence of a unified registry that can avoid these collisions and record these identifiers in a unified manner.

There are a large number of relevant standards. For example, in the field of international trade, unique product identifiers (UPIs) define the product that is sold on the world market. The manufacturer assigns the product identifiers to each item. Identifiers are used to provide device identification in different environments: Smart environment for aging; Smart agriculture and food security; Wearables and Health, Wellness; Smart cities; Smart mobility; Environment and smart water management; Smart manufacturing; Smart Energy and Smart Grid; Smart buildings and architecture; Smart homes; Smart logistics. Examples include:
The Vehicle Identification Number (VIN), ISO 3779, specifies a uniform identification numbering system for road vehicles.
The encoding, identification and marking of load containers is specified in ISO 6346.
The identification of animals with radio frequency labels is specified in ISO 11784.
The identification of RFID tags through a numbering system is defined in ISO/IEC 15963.
ISO/IEC 15459 is used for the identification of logistics items, products, returnable transport items and groupings.
Communication identifiers are essential to a communication protocol and affect its functionality (e.g. routing, switching), such as IPv4 and IPv6 address, MAC address, and so on.
User identifiers such as email address (IETF RFC 5322) and organization identifier (ISO/IEC 6523-1).
Protocol identifiers.

In addition, location identification is important in many IoT applications. There are some standards for identifying a geographic location. Examples include:
A standard representation of the location of the geographic point by coordinates, including latitude and longitude, to be used in data exchange is specified in ISO 6709.
Location identifier of the International Air Transport Association (IATA).
United Nations Code for Trade and Transport Locations, UN/LOCODE.

A common framework for identifying spatial objects or locations is another unresolved challenge. A persistent identifier (PID) acts as a standardized and long-lived invariant reference to a digital resource, regardless of its current state, location, or owner. The Spatial Object Identifier (SOID) architecture is an architecture for collecting, recording, resolving, cataloging, and disseminating PIDs associated with spatial data published by remote geospatial information systems. Today there is a large volume of spatial information published on the web, but the interfaces offered are very heterogeneous. One of the most popular interfaces is the Open Geospatial Consortium (OGC) Web Feature Server (WFS) specification. Although some states already have certain governance structures, processes, standards, guidelines and tools for managing identifiers in their Spatial Data Infrastructures (SDI), their approaches are different from each other and are at different maturity levels and at least by the applicant no common shared PID governance strategy is known for different states, there are only references to the need for a common framework. In the identification of spatial objects or locations, the persistent identifier (PID) can be identified by a unique resource identifier (URI) as UUID and also as an OID or a DID, so that it can be used by external applications to refer to it.

In addition, the relationship between identifiers can change dynamically over time (for example DNS or NDP). In any system of components that interact with each other, the identification of these components is necessary to ensure the correct composition and operation of the system. This applies to all phases of a system's lifecycle, from development to assembly, commissioning, operations, maintenance, and even end-of-life. Interoperability issues for device identifiers occur when IoT solutions have to support different identification schemes.

Artificial intelligence and robotics offer many other possibilities: camera system to see all angles; infrared and thermal night vision cameras that are able to distinguish between objects, animals, people, etc.; radars, sensors and different types of systems that allow navigation without human action, for example, to reach emergency areas avoiding obstacles. Unmanned Aerial Vehicle (UAV) is a reusable, unmanned aircraft that autonomously maintains a controlled and sustained flight level. The term UAS refers to an unmanned aerial system, such as drones. Generally they must have an identification plate, which must be fixed to the structure of the aircraft, a fire-retardant material and with information such as aircraft identification, type, model, and, where applicable serial number, as well as the name of the operator and the necessary contact details, and must be readable to the naked eye and indelible. However, at the level of connectivity between devices, there is no way this information can be associated, verified and shared by a unified registry and a unique and universal identifier, also the devices cannot automatically identify each other in a simple way neither can be located from a central control system, for example to coordinate rescue tasks in a catastrophe, nor the devices used in rescue tasks can identify devices of affected and/or missing persons.

There are, therefore, several types of identifications related to connected devices, smart devices, and spatial objects. However, there is currently no known identification scheme that can solve all the problems raised above and cover all the needs raised. IoT, robotics, and spatial object identification applications have to deal with the variety of identification schemes and interoperability issues arising from the diversity of existing identification systems.

On the other hand, within the field of biomedicine and nanotechnology, there are robotic systems in development to automate the production and analysis of mini-human stem cell-derived mini-organs. Researchers from the University Of Washington School Of Medicine in Seattle developed this new stem cell growth system into three-dimensional structures called mini-organs or organoids, which resemble rudimentary organs and, in many senses, behave in a similar way. The ability to produce organoids promises to expand the use of mini-organs in basic research and drug discovery. In addition, smart pills, also known as capsule endoscopes, can be located in the body using magnetic fields. The technique uses silicon chips that contain a set of integrated sensors, resonators and wireless transmission technology that allows them to mimic the magnetic resonance properties of atoms to be detected by Magnetic resonance imaging (MRI).

Communication between microscopic robots (nanorobots) can coordinate activities for biomedical tasks. The feasibility of in vivo ultrasonic communication for micron-sized robots that transmit to various types of tissues at frequencies between 10 MHz and 300 MHz, offers the best compensation between efficient acoustic generation and attenuation for communication at distances of approximately 100 microns, so that the power available from ambient oxygen and glucose in the bloodstream can readily support communication rates of about 10,000 bits/second between micron-sized robots, without damaging nearby tissue.

The application of devices and sensors implanted within the human body to transmit health data of patients can provide great advantages in continuous medical monitoring. The use of RFID for in vivo data collection and transmission has been successfully tested for electroencephalograms. Acoustic, light, RF and chemical signals can be considered as possible options for communication and data transmission for communication in liquid workspaces, depending on the application.

The architecture of a nano-robot can include integrated nano-electronics and the use of mobile phones for different diagnoses, eg. for the smart distribution of chemotherapy drugs and the redetection of tumors for cancer treatment. The nano-robot can use an RFID CMOS transponder system for in vivo positioning, using communication protocols that allows information to be tracked about the position of the nano-robot. This information can help doctors to detect small malignant tissues even in the early stages of development. The outer structure of the nano-robot may be composed of materials that ensure sufficient biocompatibility to prevent the immune system from attacking.

Nano-robots that use programmed chemical nano-biosensors to detect different levels of E-cadherin and beta-catenin in the primary and metastatic phases can detect and identify changes in Ecadherin protein gradients beyond expected levels, which can guide nano-robots to detect tumor cells in the early stages of cancer. There is a wide variety of possible sensors; for example, chemical detection can be highly selective in identifying various types of cells using markers. Chemical signals and interaction with the bloodstream is a key aspect of addressing the application of nano-robots for cancer therapy. Acoustic detection is another possibility, using different frequencies to have wavelengths comparable to object sizes of interest.

An important aspect is that robots and other medical devices have to comply with a specific identification. There are different UDI (Unique Device Identification) systems for medical use in different territories (Europe, USA, . . . ), but these systems are independent and isolated from each other. The FDA (Food and Drug Administration) in the U.S. implemented its UDI system in September 2014 for medical devices. Subsequently, European Regulation 2017/745 on medical devices established its own UDI system, which aims to improve the quality of information in medical devices through a European database for the identification of these medical devices (EUDAMED).

In this regard, application U.S. Ser. No. 16/169,126 (PCT/ES2019/000062) establishes a universal registration and identification procedure for products of biological origin for medicinal purposes to unify the different UDI identifiers; the identification code according to the description of that invention contains product identifiers from one or more countries, one or more product labeling systems and, in addition, one or more links to web addresses, and the procedure generates a global data vault for each product, different local data vaults of the same product for each specific data legislation and a public and private identifier associated with the global vault.

The U.S. Ser. No. 16/452,598 application uses the foregoing and extends it to describe a device identifier (DEV) called X-UHC-DEV that can be associated with both an individual's device and a medical, veterinary or enterprise device, among others, and uses the decentralized blockchain principle with identity verification in join with a unified and universal multipurpose identifier for different uses such as for an individual, established relationships with the individual, devices, products and services, transactions as access permissions granted/revoked by the individual, among others.

The object of the present invention is to improve the current state of the art and solve the different problems and needs posed, through a procedure that allows the unified global registration and the universal identification of spatially locatable objects using UUID, OID and DID identifiers, without discarding others, and expanding the identification systems known so far to achieve unification in all systems and territories of identifying these objects, improving the care and support to people in their health treatments as well as in emergencies and disasters mainly, without excluding other uses, as well as facilitating the provision of services.

Object identifiers or OIDs are an identifier mechanism standardized by the International Telecommunication Union (ITU) and ISO/IEC to identify any object, concept or “thing” with a globally unambiguous persistent name. OIDs are paths in a tree structure, where the leftmost number represents the root and the number on the right represents a leaf. Each OID is created by a Registration Authority. Each of these authorities may, in turn, delegate the allocation of new OlDs to other registration authorities, and so on. A leaf node can represent a registration authority (in which case the OID identifies the authority), or an instance of an object. Once issued, an OID is never removed and always identifies the same schema or object. For example, 1.3.6.1.4.1.19126.3 refers to the National Identity Document in Spain (DNI), OID number 2.16.724.4.302 refers to the number of physician collegiate in Spain (College of Physicians) and there are also OID numbers for different Spanish administrations.

A UUID is a universal unique identifier, standardized by the Open Software Foundation (OSF) as part of the Distributed Computing Environment (DCE), which is unique in space and time with respect to the space of all UUIDs by using cryptographically secure random numbers, used to uniquely identify an object or entity on the Internet. UUIDs are represented as a URI or uniform resource identifier and refer to an internet resource; it can bring additional data to indicate how to access the resource, for example. URN (Uniform Resource Name, IETF RFC 2141) is a uniform name for resources; it indicates the name of a resource that must be unique across the Internet. A URN formed using a UUID will be of type “urn: uuid:” followed by the hexadecimal representation of a UUID. In addition, a UUID can also be encoded as an OID.

On the other hand, verifiable claims are a standard defined by the World Wide Web Consortium (W3C) and are defined as a characteristic, property, attribute, or background information of an entity (such as name, identification number, current and former owner, model, etc.) and provide a standardization and interoperability mechanism for the purposes of storing, transmitting, and receiving digitally verifiable attribute receipts, such as providers, manufacturers and owners of goods and/or services. A decentralized identifier (DID) is a new type of global unique identifier, under the World Wide Web Consortium (W3C) standard, and is defined as “a new type of identifier for verifiable and self-sovereign digital identity”. It is a multi-attribute schema that uniquely defines a person, object, or organization. Each DID record is cryptographically protected by private keys and can be adapted to work by the same basic pattern as the URN specification (urn:did). DIDs provide a path to digital data portability with verifiable credentials, and all types of identifier systems can add support for DID. This creates a bridge of interoperability between the worlds of centralized, federated, and decentralized identifiers.

A DID is two things: a unique identifier and an associated DID document. A DID document is a JSON-LD object that is stored on any computer system for easy querying in a “persistent and immutable” manner. The content of a DID may be cryptographically signed to ensure its integrity. Using cryptographic keys, the owner can prove their authenticity. Parties that have exchanged DID can authenticate each other and encrypt their communication. The DID document can include a timestamp of when it was created, a cryptographic proof that the DID document is valid, a list of public cryptographic keys, a list of ways the DID can be used to authenticate, a list of services where DID can be used, and a number of externally defined extensions. Also, any information can be referenced from service endpoints. Through this mechanism, DIDs provide a permanent and resolvable identifier for any Internet service. DIDs represent a universal layer of addressing, abstraction, and verification for key pairs, endpoints, and any other information included in the DID document.

Regarding communication devices, the MAC address is a unique identifier that manufacturers assign to a network card or device. It is also known as physical address, and theoretically it is unique worldwide and fixed for each device. It is important to note that a computer can have varied hardware to connect to networks; in this way, it is common to have one MAC address for Ethernet, another for WiFi and another for Bluetooth.

Currently, 5G mobile technology brings very significant benefits due to its low latency, allowing data and instruction communications to be virtually instantaneous. However, in disaster situations there may be a drop in telecommunications networks, such as mobile telephony and therefore 5G support, so additional technologies such as Bluetooth, Wi-Fi, Wimax or LPWAN may be required, although LPWAN has many limitations, as an example in some countries only 1% of the time can be emitted and the transmission speed is very low. Therefore, new solutions are necessary to address these problems.

Bluetooth is a collection of different protocols grouped into a single specification. The Bluetooth interface, while on and not linked to any device, periodically sends the UUID, which is the universal unique identifier and can be tracked, to “neighbors” devices. A beacon is a small, energy-efficient device that can be powered by a battery for a long period of time (up to several years), and emits a broadcast signal via low-power Bluetooth connection (BLE). Beacons transmit a signal with a fixed power, known as Tx Power. As the signal travels in the air the signal strength decreases with the distance. The low energy signal can be captured by nearby mobile devices (smartphones, smart-watches, . . . ) enabled for Bluetooth and detected by an application on the mobile device. The application can then process the captured signal from the beacon and transmit it to a cloud server over the internet. Based on the signal strength of the beacon received on the mobile device, the user's distance to that beacon can be calculated, without the need to use GPS, for locating devices with a much lower power consumption and therefore a greater autonomy. For example, it can be used to locate checkpoints, for signage and spatial locations, as well as indoors (e.g. in hospitals), where there is usually no GPS signal coverage, for locating people (patients and professionals, doctors, nursing, civil protection, police, volunteering, etc.), vehicles (such as ambulances, extinguishing equipment, drones, etc.) or devices such as medical devices, smartphones and other electronic devices. The cloud server can then process this information and perform more detailed analysis based on the specific location of the user's device.

There are two main roles that beacons can play: peripheral and central. The purpose of peripheral devices is to stay in sleep mode for as long as possible by saving battery and periodically sending data to a central device. A central device has much more processing power and acts as a cloud or internet link device. There are different protocols. For example, the Eddystone specification allows to know the health status of the beacon, such as the level and battery life, which also allows the monitoring of beacons from the cloud server from the application installed on a smart device. Currently, there are on the market Bluetooth Smart sensor beacons with a range of up to 1 kilometer in the open field and an estimated battery life of 10 years and even longer.

On the other hand, with regard to the identification of individuals and related organizations, it should be noted that patent ES2615815 (PCT/ES2016/070428) allows a unified registration of blood donors (and other types) and the unification of different identification cards for the same person in the field of donation. This universal health identifier is used in the U.S. Ser. No. 16/169,126 (PCT/ES2019/000062) application for the universal identification of individuals in the field of health, as well as in the U.S. Ser. No. 16/452,598 application to establish a unified identification protocol in training and health through a multipurpose universal identifier for individuals, organizations, services and devices (among others that is proposed by such invention), and which can serve as a basis for achieving the objectives that the present invention proposes. Additionally, the Legal Identity Identifier (LEI) and Business Identifier Codes (also known as BIC or SWIFT) can be used to identify legal entities in financial transactions.

In conclusion, and as a reference to the current state of the art, it should be noted that, although different standards, types of registration and identification systems are known on the market, at least on the part of the applicant, the existence of any one with technical and constitutive characteristics similar to those presented by the invention that is asserted here is unknown.

### SUMMARY OF THE INVENTION

Small technology (microelectronics, microsystems, micro-sensors, nanotechnology, smart tags, . . . ) together with the dissemination of wireless networks and connections of point-to-point devices (wireless, WLAN, Bluetooth, infrared . . . ), allow the existence of very small interconnected computers without cables that integrate almost invisibly into any type of object. Using small sensors, these built-in processors can detect the environment around them, giving the object itself the capabilities of processing information and communication. It is the complete computerization and interconnection of objects or “things” and also implies the possibility of connecting almost anything to the Internet. The development of wireless devices, modules (integrated in a solution that builds new embedded devices) and terminals (independent units that can be connected to other devices) can transform almost any object into a network-connected object. This increases the degree of communication and cooperation with other “connected” objects, which can be considered as “smart” if they extract knowledge from certain data to improve their response (without becoming “intelligent beings”), and which bring multiple possibilities of connection, communication and service to all individuals and systems that use them.

A device called “smart” is therefore a device equipped with some processing capabilities, usually with the ability to connect to other devices or networks through different communication protocols (Bluetooth, NFC, WiFi, 4G, etc.), and that can work to some extent interactively and autonomously. It may also refer to a ubiquitous computing apparatus that exhibits some properties of ubiquitous computing, such as artificial intelligence.

Ubiquitous computing (ubicomp) refers to the integration of computing into the person's environment, so that computers are not perceived as distinct objects. It can also be referred to as environmental intelligence and, from the point of view of the actors, it is also known as elements of the Internet of Things (IoT), haptic computing, and even artificial intelligence. The Internet of Things (IoT) is a scenario in which objects, animals or people have identification and the ability to automatically transfer data over a network, without the need for human-to-human interaction or human interaction thanks to the convergence of wireless technologies, micro-electromechanical systems (MEMS) and the Internet.

A robot is a type of physical artificial device (electromechanical, biomechanical . . . ) or virtual (these are usually referred to with the term bots) that is considered “smart” for its processing and connectivity capacity. In practice, it is usually an automatic and programmable device or system capable of performing certain operations autonomously; it can be equipped with sensors that allow it to adapt to new situations and also be connected with other devices or networks; may be able to replace human intervention in certain tasks, as well as make it easier for humans to perform certain operations, such as surgical interventions or remote object handling, for example.

On the other hand, spatial object is defined by INSPIRE (European Commission) as the abstract representation of a real phenomenon corresponding to a specific location or geographical area. Localization, understood as the relationship between a concept that is located and a geo-referenced place or spatial object (a set of coordinates), is a common form of spatial reference.

The concepts that are located are defined in the present invention as spatially locatable objects and can be directly related to both connected and/or smart devices as well as spatial objects, and indirectly with individuals, organizations, services and others. The spatially locatable object can be of one or more types: a spatial location (physical object represented by a set of coordinates or defined zones, for example by a vector map) which can incorporate connected or smart devices to be provided in this way with digital services; also a connected or smart physical object (e.g. a RFID or Bluetooth beacon, a Smartphone, . . . ) or a robot (electromechanical, biomechanical, autonomous, remotely operated, . . . ), without ruling out others.

The spatially locatable object can therefore be identified by a set of coordinates or vectors fixed or variables, can contain or be defined by one or more connected and/or smart devices and, in turn, may therefore be contained within another object (root or parent object) as well as contain other objects (child objects). As a non-limiting example, the following can be cited: a smartphone is a spatially locatable object via a mobile phone connection, a WiFi connection, Bluetooth or others; a certain area within a hospital is a spatially locatable object that can be referenced by predefined coordinates or by one or more Bluetooth or RFID beacons and it may have associated different medical devices as well as smartphones of patients and/or family; a medical nano-robot is a spatially locatable object in a certain area in a patient's body and also in a certain area of a hospital (laboratory, operating room, . . . ); an ambulance is a spatially located vehicle within another such as a road, and this in turn may contain other spatially locatable objects such as connected or smart signaling devices (e.g. Bluetooth beacons or RFID devices). Furthermore, the procedure explains that an authorized human user through an application and a computer system, an authorized service, an authorized spatially locatable object such as a smart device, a connected machine, among others, can send data and instructions to one or more spatially locatable objects to establish actions and coordinate operations.

The present invention further states that 1 spatially locatable object may contain information such as a list of objects and individuals that have been authorized and/or have accessed said object or its services, accesses made to the different services and/or to different types of information of the object (such as public, private, emergencies, regulatory information), date and time of said accesses, among other possible information; the authentication can be made through multifactor authentication and the multifactor authentication can be made by reading a PIN code, QR or NFC codes and/or biometric data among others, being the multifactor authentication first done in a concrete spatially locatable object and then in a spatially locatable object of an individual which can be also related to the first spatially locatable object; for example, the access to a spatially locatable object such as a certain restricted area in a hospital can be made through both a peripheral security device which is part of that restricted area (child object of that spatially locatable object) and a pre-authorized smartphone of an individual in that spatially locatable object (also considered as a child object of the first spatially locatable object).

A spatially localizable object may contain information on the processes carried out and different information on these processes such as who made a request to the object, who authorized it (if approved was required), participants, tasks performed and results, products obtained or manufactured (if applicable), among other possible information; these processes are referenced to information inside or outside the DID document as public, private, emergency, and/or regulatory information, among others, and can be linked to different identifiers such as medical process identifier, biological product identifier, without ruling out others.

The invention of the present descriptive memory refers to a procedure for the unified registration and universal identification of spatially locatable objects (such as smartphones, smart-watches, IoT devices, robots, buildings, vehicles, roads, rivers or mountains, for example), as well as related individuals and organizations and also associated insurance in each case (e.g. health, life or other); the purpose of this invention is to achieve the unification of the identification of these objects in all systems and territories, aimed mainly at, without excluding other embodiments, improving care and support to people in their health treatments and in emergencies and disasters, as well as facilitating the provision of services.

The objective of the present invention falls, in particular, on a procedure whose purpose is to achieve, as already mentioned, the unification in all systems and territories of the identification of spatially locatable objects, both of devices and of spatial objects or locations, as well as related individuals and organizations and associated insurance data in each case, for the best care and provision of services to individuals such as emergencies and disasters, without ruling out others, in order to facilitate access to information and localization in a unified manner between different systems, through appropriate access to such information, as well as to facilitate personalized care, support and treatment for individuals, consisting such identifier in a code in URN UUID, OID and DID format (without discarding others), which is deposited and stored in the database of a computer system accessible by the different authorized parties in one or more territories.

The invention therefore proposes the development of a unified registration and universal identification procedure that allows:
Register and identify or locate spatially locatable objects using a unique, non-transferable identifier.
Manage the life cycle of spatially locatable objects.
Achieve data interoperability between different identifiers and systems, to identify spatially locatable objects as well as users and organizations related to them, and also the associated insurance.
Obtain the identification and location of objects and devices in a certain area quickly and conveniently, as well as individuals and other data related to them, at any given time.
Facilitate the identification and location of these objects and devices and also of individuals in case of emergencies and disasters, as well as the access to the data.

The present invention presents advantages and characteristics, which will be described in detail below, which represent an improvement of the current state of the art.

### DESCRIPTION OF THE DRAWINGS

The achievements in this document shall be better understood from the following detailed description with reference to the drawings, in which:

FIG. 1 shows, using a block diagram, the operating flow scheme of the procedure, according to the invention, for unified registration and universal identification in at least two operational phases.

### DETAILED EXPLANATION OF THE INVENTION

The proposed invention refers to a procedure for the unified registration and universal identification of spatially locatable objects to achieve unification in all systems and territories of the identification of these objects and facilitate access to data related to them, such as spatial locations, individuals and organizations related to them and also associated insurance in each case, to improve the care and service delivery to people, as well as to serve as a utility in emergencies and disasters, without excluding other possible uses.

The application U.S. Ser. No. 16/452,598 describes a protocol to universally identify an individual, organization, service and device (among others) for training and health through a multipurpose universal identifier, and also establishes how to access such data anywhere upon authorization. Through its utilization, the herein proposed invention may make the universal identification of individuals, organizations, services and devices, not only for training and health but also for other uses, and therefore can be universally carried out the identification of those (individuals, organizations, services and devices) related to a certain spatially locatable object, as well as obtain access rights to certain data of an individual, organization, service or device related.

The purpose of the present invention is, therefore, to facilitate the registration and identification of these spatially locatable objects (such as roads, hospitals, housing, smartphones, tablets, smart watches, IoT devices, smart land and air vehicles, and others) to achieve interoperability between spatial locations, different types of devices and computer systems in any territory, in order to facilitate access to the information and location of the object in a unified manner as well as the assistance and provision of services, carried out through the uniformity of both identifiers, registration and access to the data of these objects, and also of the individuals and related organizations; it also allows to know certain data both of the object (e.g. X-UHC-DEV identifier, local spatial object or location identifier, areas with which it is associated or in which it is located, licenses, manufacturer, model and serial number of the manufacturer, legal registration of the object, transfer of ownership, user, insurance, proof of payment, services provided, among others) and of the owners or associated users (such as multipurpose identifiers of individual and organization, tax identifier, health insurance, identity document, without discarding any additional data); consisting of such a procedure in a spatially unique and non-transferable locatable object identifier, performed through the use of UUID, OID and DID formats (without discarding others); this identifier is deposited and stored in the database of a computer system accessible by the different authorized parties from one or more territories by establishing and controlling the access rights that are necessary for each case; such identifier has certain information associated with it such as public, private, regulatory and/or emergency information, for example identity registered with the regulator on which it depends, identifier and data of an associated insurance (eg. period of validity and proof of payment), model or type of object, other identifiers such as those mentioned above and also identifiers of its components (memory module identifier, MAC identifier of the WiFi and Bluetooth card, UUID identifier of Bluetooth or RFID, . . . ), among other possible.

On the other hand, biological drugs and other medical devices have some potential to be recognized by the body as foreign substances and, consequently, have the inherent ability to induce unwanted immune reactions. In the case of smart biomedical devices such as smart pills and others, these can be created specifically for the treatment of a particular individual, for example. The U.S. Ser. No. 16/169,126 (PCT/ES2019/000062) application establishes a unified global registration and universal identification of products of biological origin for medicinal purposes, as well as the storage of public and private data of the product, so that in the case of smart medical devices such as nanorobots that have been manufactured with products of biological origin, such invention will be used within the invention proposed here for the registration and identification of products with a biological content for medicinal purposes, establishing a unique relationship in the system of the invention that is proposed between the spatially locatable object identifier and the biological product identifier for medicinal purposes.

Through its correct implementation, the invention proposed here allows to know the identification and access to certain data of the different spatially locatable objects, for example vector data of the object, legal or regulatory, property, data of individuals (such as owners or users), organizations, devices, services, payments and related insurance, among others, carried out independently of the type, model and technology of the object as well as the manufacturer, regulatory body, type of connectivity that it uses and others, either prior to a request for care or the initiation of an assistance or during and after it, to assist both in the care and support of the individual and in the provision of services and the realization of certain operations.

Thanks to the communication systems (WiFi, Bluetooth, RFID, 5G, etc.) sensors integrated into robots, smartphones, roads and vehicles, among others, are able to communicate with each other and through the invention that is proposed can perform the identification of a spatially locatable object (e.g. near or detected) quickly and practically, and access and/or request access to the data related to it. For example, medical nanorobots, connected vehicles, drones and others can improve their operation in a practical and effective way.

As described, the proposed invention therefore allows the unified and universal identification of spatially locatable objects, as well as the entities and individuals related to these and the insurances associated in each case, for better attention and provision of services. Therefore, through the unified registration and the identifier that the invention proposes, a spatially locatable object can automatically detect the presence or existence of others in a nearby environment (within a radius of action) to a specific area or route, allowing to generate a notification to the different related parties and including the possibility of connection between them, by exchanging their identifiers to send notifications and/or instructions in one direction (one-way communication) or bidirectionally for the transfer of notifications, data and instructions between them.

By way of non-limiting example, by using the procedure this invention proposes, it is possible to make the warning of the need to pass an emergency vehicle on a certain route even before starting the journey, both to autonomous vehicles such as smartphones located or close to that route, so that they can leave free passage even before the emergency vehicle is detected by its sound signals, and can know in real time the expected time of arrival, alerting drivers and to police to this (e.g. traffic management).

Also as a not limiting example of what this invention proposes, by using the spatially locatable object identifier either in a crashed vehicle or in an app on a smartphone or smartwatch, and using Bluetooth technology and/or others such as 5G or WiFi, the spatially locatable object can be used as a beacon before, during and after an emergency or catastrophe, so that an emergency, civil protection, firefighter, police, etc. service can detect the signal object and/or position of the object and uniquely identify the object and also the individual or individuals related to or associated with it, and give notice where appropriate to a medical service and the corresponding insurance; all this carried out through the purpose of the invention for the unified global registration and the universal identification of spatially locatable objects and the different related parts such as, for example, the Bluetooth communications card of an individual's smartphone/smartwatch which is associated with the spatially locatable object identifier in the database of the computer system of the invention proposed and with the X-UHC-DEV device identifier of the invention U.S. Ser. No. 16/452,598, and this spatially locatable object identifier will in turn be related to the individual's multipurpose identifier, so that emergency data that a user has shared for that purpose can be accessed through the individual's multipurpose identifier (e.g. blood group, emergency contacts, and other relevant medical data).

A broader example of what this invention aims to achieve, is described below: The spatially locatable object identifier is transmitted, from a smart device of the user (e.g. smartphone, smartwatch or smart vehicle) and by authorizing it, to one or more databases in the cloud (distributed and/or centralized), during regular periods of time, which perform the collection of this data for prevention and use in cases of emergencies and disasters, together with the location of the device and, in addition, of the related entity(s) or individual(s). At a certain point, a serious incident (such as a fire, earthquake, tornado, or flood hazard) is detected by the civil protection services in a given area. Using the spatially locatable object identifier, the civil protection service can query the database accessible to the different authorized services, filtered for its coverage area or consultation permit and/or for the affected area or related spatially locatable objects, locate on a map the distribution of the population, identify the devices associated with individuals in that area, as well as citizens, volunteers and professionals (medical and nursing personnel, firefighters, civil defence, civil protection, etc.). The civil protection service can send an alert to objects located in that area, depending on the type of object, to take different actions and monitor in real time the movements and displacements of the population and professionals. In this way, the invention allows help in decision-making and better management of resources, prevent and inform the population and take appropriate actions. It also allows the location of affected and/or survivors to be detected either by GPS geolocation, WiFi, LPWAN, Bluetooth or others, for example through manned reconnaissance flights (such as helicopters or light aircraft) or unmanned ones (drones and others), obtaining the identifier of the detected spatially locatable objects (from the Bluetooth UUID of those affected, for example) and consulting certain information about those identifiers in the accessible database according to the invention, such as the identifier corresponding to a Bluetooth device, entities and associated individuals, as well as other relevant data, such as medical data for emergencies.

Other achievements are also available for accident prevention. For example, the different driving assistance programs (maps) can know in a unified way the vehicles driving on a spatially locatable object, such as a road, regardless of the navigation system, the manufacturer or the type of vehicle. In addition, by incorporating technology such as bluetooth or others, beacons may be included in traffic signals, electrical poles, along the roads (signaling of kilometer points) or others, to be detected by the devices in-vehicles and know the location of the vehicle and other nearby vehicles, visually indicating the presence of vehicles or others that may interfere with the route (e.g. at a junction) or vehicles to the contrary (e.g. on road dual-way with little visibility).

Another use case that this invention proposes is to know, on the part of the civil protection services and insurers among others, the owners and inhabitants in areas affected by catastrophes, needs of medical conditions according to the emergency data of the affected individuals, goods affected in such areas and insurance data of both individuals and goods. By using an app on users' mobile devices, users can use a connection (Bluetooth, WiFi, LoRa networks, 3G/4G/5G or others) to send their state (trapped, injured, pain in certain parts of the body, etc.) and could share relevant medical data (medications, diagnosed diseases, etc.) to perform a medical triage from an emergency center and better manage the care and resources available. Similarly, through an application for this purpose, emergency services can give advice to the population located in a certain area or route, notify field hospital locations, drinking water points and even organize delivery of medicines and food through vehicles (terrestrials, drones, etc.) to the locations of affected people.

Going into more detail, it should be noted that the procedure of unified global registration and universal identification of spatially locatable objects will generate a unique and non-transferable public identifier preferably in a UUID format, be recorded in at least one OID, have an associated DID and DID document, and this identifier as other identifiers and related data will be stored in one or more centralized and/or distributed databases. The DID document shall contain public, private and, optionally, information from the relevant regulatory authority. For example, if it is a spatial object such as a property (housing, land, garage, . . . ), the associated DID document may contain location information, such as address, geographic coordinates and cadastral reference (regulatory body data), owner and user (e.g. tenant and other people who may be living or registered in that dwelling), property insurance data (insurer, period and payment information, among other data), plans and other information about it (height, square meters, common areas, etc.), materials contained and/or insured, belonging to another locatable object (e.g. a building, street, locality . . . ), without discarding others. The DID document will also contain a list of services and the corresponding URIs in HTTP format to access both the data and services related to the generated identifier.

It should be noted that this public identifier can be marked in disuse and also replaced by a new one, marking the old deprecated one and pointing to the new one if applicable, varying the associated DID identifier and/or DID document, so that it can continue to be referenced and accessed and not reused for another object. For example, in case of change of owner or user, the public identifier can be changed by the regulatory body (just like the license plate of a vehicle is associated with a chassis number, for example). If the object or device belongs to another spatially locatable object, the transaction made at the time of the change of identifier will notify related objects, individuals, and organizations of such change. The old identifier will be deprecated and can be redirected, for example, to the new identifier or to the competent regulatory authority.

Private information may contain both associated user(s) or owner(s) information and information from different identifiers associated with the object. For example, in the case of a spatial object such as a specific area within a building may contain a list of allowed or authenticated users, also other related spatially locatable objects; in case of a robot, connected or smart device, private information may contain different associated identifiers such as product identifier and different components (Bluetooth, Wi-Fi, etc.) and also other spatially locatable objects.

Such identifiers, as well as the data associated with the identifiers in the DID document and the access rights to such data (public, private, emergencies, regulatory, . . . ), will be certified in one or more DLTs or blockchain networks to ensure the immutability of the data. The DID document may refer to other documents with public, private, emergency or regulatory information, which can also be stored on one or more DLTs or blockchain networks. The different authorized parties will be able to access public data as well as private, emergencies and regulatory data, depending on the permissions defined in the system through the DID document and the certification of its validation using blockchain technology, without ruling out other alternatives. This is done through an access control list or ACL, where the different permissions are collected for each user and group of users (individuals/organizations or groups of individuals/organizations). By calculating a hash function, the contents of the information contained in the DID document (data, permissions, document content, etc.) will be sealed to ensure the unalterability of the data on one or more blockchain networks, so in that way it can be verified that the data contained and retrieved from the DID document are true, are not erroneous and have not undergone any unauthorized modification. In case of the hash not matches with the received data, the different parties will be notified of such a situation to take the required actions in order to solve the problem.

The procedure of unified global registration and universal identification of spatially locatable objects and the different related parts employs preferably a unified protocol of identification in training and health in join with the unified registration and identification of donors, Legal Identity Identifier (LEI) and Business Identifier Codes (BIC) for the universal identification of individuals, organizations, services and devices, for data verification and accessing relevant data among others; also the unified registration and universal identification of biological products for medicinal purposes to identify spatially locatable medical devices containing biological product derivatives and the related parties.

The proposed invention is therefore configured as a novelty within its scope, since according to its implementation the above objectives are satisfactorily achieved, being its details characterizing conveniently collected in the claims.

The invention described essentially comprises the following:
Generation of a unique, non-transferable identifier for each spatially locatable object, in URN UUID, OID and DID formats, without discarding others.
Creating a DID document associated with the generated identifier.
Association of different object and user/owner identifiers.
Assignment of permissions and creation of access control list (ACL).
Creating encryption keys associated with the ACL.
Save in the DID document data, service identifiers, and other associated identifiers, as well as permissions required for access to object data.
Digital fingerprint generation using hash-algorithm to seal data and identifiers and perform digital signature.
Storage of both associated data and hash in one or more blockchain networks.

It should be noted that the universal identifier of the object is either generated by an organization or a user through an application or a service, or a regulatory body also through an application or a service, and through a computer system specifically configured for this purpose.

This allows, on the one hand, the unified identification of spatially locatable objects by agencies and users (humans or services) with permission to do so and, on the other hand, the unified identification by other spatially locatable objects (e.g. nearby smart devices or other related objects) as well as knowing the location of the objects. By means of the data collected in the DID document associated with each spatially locatable object and the corresponding necessary permissions, can be accessed the corresponding details of each specific object. In addition, a notification may be sent to one or more related objects, individuals and/or organizations where relevant information exists.

If the DID document contains data that has been added by an individual/organization, it may optionally be verified that the data entered are real, for example after notifying and/or capturing information from different organizations such as manufacturers and regulatory bodies, among others, and updating the data collected in the DID document as verified data, for example by a regulatory body with permissions to modify the object data.

In the light of the FIGURE described, and according to the numbering adopted in it, one can see how the invention comprises, essentially, the following phases and steps:

In a first phase (A):
(i) generation of a universal, unique and non-transferable identifier for a spatially locatable object in URN UUID, OID and DID formats or in other embodiments, through an application or service and a computer system configured for that purpose;
(ii) generation, through the computer system, of a DID document associated with the DID identifier, containing a list of permissions (ACLS) and services, associated data (public, private and, in addition, one or more regulators) and hashing of the data;
(iii) storage of the data and hashes associated with the DID document in one or more centralized and/or distributed databases (DLT or blockchain);

In a second phase (B):
(iv) identification of the object in the system or any of its components (such as Bluetooth, WiFi, space object or other communication devices) by means of the identifier generated for access to the details of the same, made by another object, a individual or other system, through an application or service for this purpose;
(v) identification of the different present or detected objects in a given location and/or related to another object, by means of the universal identifier generated in the system for each of them;
(vi) send the result of the identification of one or more objects through a communications network.

In addition, optionally, the invention contemplates, in the phase of use:
vii) Request data and/or list of services directly to the object;
(viii) receive data and/or list of services from the object.
(ix) Request data related to an object from external data sources (such as manufacturers or regulatory bodies, among others);
x) receive data related to an object from an external data source.

Optionally, the invention also includes:
xi) automatically detect the presence of a spatially locatable object (within a radius of action);
(xii) send a notification to an object and to a user, owner or organization related to it, indicating the reason and urgency.

Finally, optionally the invention also includes:
(xiii) verify that the data related to a given object is real after obtaining data from a reliable external data source (e.g. a regulatory body) and/or have been verified on one or more DLTs or blockchain networks;
(xiv) establish a connection to the object once the authenticity of the object data has been verified.

The described unified identifier of spatially locatable objects is therefore constituted in an object of characteristics unknown until now for the purpose of which it is intended, reasons which together with its practical usefulness, give it sufficient foundation to obtain the exclusivity privilege requested.

Each of the steps described herein can be performed using a general purpose computing device, e.g., a personal computer, personal digital assistant, and/or computing device that is in communication with a network, e.g., internet, intranet, or extranet, that includes a memory and a set of instructions, that is, logic, specifically scripted to perform the functions, steps, communications, and data manipulations described herein. As the present invention is not limited to any specific instruction set, e.g., code, usable to implement one or more aspects of the present invention, and as those skilled in the art are well aware of the ways to instruct such a computing device to implement such functions, steps, communications, and data manipulations, further details of such specific instruction sets will not be provided here so as to not obscure the present invention. While one or more of the functions, steps, communications, and data manipulations can be performed by hand or by a computer-implemented instruction set, the present invention is not limited to any one sub combination thereof. Data gathering can be performed manually, in a semi-automated manner (e.g., given to a human person who then inputs the data into a computing device's memory), or in a fully-automated manner (e.g., the customer interacts directly with a computing device to input the data into a computer's memory). As will be readily appreciated by those skilled in the art, the present invention also includes the use of multiple computing devices on a communications network (including the use of removable memory media to transfer data between devices), including that the computing device via which data is input by any one human user is different from a computing device that implements decision instruction sets.

However, it should be understood that although numerous characteristics and advantages of the present invention have been exposed in the above description, along with details of the structure and function of the invention, this exhibition is only illustrative. Changes to the details may be made, especially in matters of the shape, size and arrangement of the parties within the principles of the invention to the extent indicated by the broad general meaning of the terms in which the enclosed claims.

Sufficiently described, the nature of the present invention, as well as the way in which it is implemented, it is not considered necessary to make its explanation more extensive so that any expert in the matter understands its scope and the advantages that derive from it, stating that, within its essentiality, it may be put into practice in other modes of realization that differ in detail from that indicated by way of example, and to which it will also achieve the protection that is sought whenever it is not altered, modified or modified its fundamental principle.

What is claimed is:

1. A procedure of unified global registration and universal identification of spatially locatable objects and the different related parts, wherein it includes: a computer system composed of computing processors, operational computer-readable memory, databases for data storage, network and display communication devices, configured to generate a unique and non-transferable identifier of spatially locatable objects;
and two operational phases A and B;
In a first phase (A):
(i) generation of a universal, unique and non-transferable identifier for a spatially locatable object in URN UUID, OID and DID formats or in other embodiments, through an application or service and a computer system configured for that purpose;
(ii) generation, through the computer system, of a DID document associated with the generated identifier, containing a list of permissions (ACLS) and services, associated data, and hashing of the data; and
(iii) storage of the DID document, the data in the document and the hash associated with the document in one or more centralized and/or distributed databases;
In a second phase (B):
(iv) identification of the object or any of its components (such as Bluetooth, WiFi or other communication devices) by means of the identifier generated for access to the details of the object in the system, made by another object, an individual or other computer system, through an application or service for this purpose;
(v) identification of the different objects present or detected in a given location and/or related to another object, by means of the universal identifier generated in the system for each of them; and
(vi) send the result of the identification of one or more objects through a communications network.

2. The procedure according to claim 1, wherein the spatially locatable object identifier is uniquely associated with a DID document and also associated with other object identifiers, for example because the object is part of another object or because the object contains others (child objects); additionally it can be associated with one or more local identifiers for one or more different regulators and/or territories, manufacturers or standards, among others; the identifier has one or more states such as in force, expired, revoked, withdrawn or rejected, without discarding others.

3. The procedure according to claim 1, wherein all or part of the object information is contained within the DID document and the DID document contains one or more identifiers of individuals, organizations and/or entities related to the spatially locatable object; another part of the information on the object is available through a service provided by the object itself and also by an external service such as a regulatory body or through one or more DLTs or blockchain networks.

4. The procedure according to claim 1, wherein the DID document contains: public, private, specific information for each of the regulatory bodies in each territory and/or for emergencies, without discard any possible ones; a list of services and the corresponding URI addresses in HTTP format to access the services, as well as information about the object such as object location (e.g. coordinates, vector map or cadastral reference), data on individuals and related organizations (owners, tenants), insurance related to the object (insurer, proof of payment, period), change of ownership, access permissions, among other data; the content in the DID document is digitally sealed by calculating a hash function and storing the hash value obtained in one or more DTLs or blockchain networks, in a non-limited manner, to certify that the content of the DID document has not been altered or corrupted.

5. The procedure according to claim 1, wherein it uses preferably: the unified protocol of identification in training and health in join with the unified registration and identification of donors (blood, plasma, platelets and other types), Legal Identity Identifier (LEI) and Business Identifier Codes (known as BIC or SWIFT) that are used for the universal identification of individuals, organizations, services and devices, for data verification and accessing relevant data, among others; also the unified registration and universal identification of biological products for medicinal purposes to identify spatially locatable medical devices containing biological product derivatives and the related parties.

6. The procedure according to claim 1, wherein the communication of data with the computer system and with the objects being carried out on a secure network (VPN, for example) or on an unsecured network such as the Internet using a secure communication protocol such as HTTPS; HTTP headers are used to send information such as the identifier of the objects and JWT tokens; JWT tokens are used as one generated after a successful authentication and that optionally contains roles and permissions after authentication is verified; additionally, for the secure exchange of data is used: decentralized identity (e.g. Hyperledger Indy or Aries), certificates such as those generated by Hyperledger Fabric, symmetric or asymmetric key encryption (public and private key pair), among other possible; the access to certain data such as private, regulatory or emergency data is made by using a permissions control with an access control list (ACL) and a role-based access control system (RBAC).

7. The procedure according to claim 1, wherein it also includes:
(vii) send data to an object (informative, operations to be carried out, updates or other) or request data from an object (informational, list of services of the object or others), either directly to the object or through the system, from organizations or individuals that allow it to be carried out (owner, regulatory body or others);
(viii) receive data from the object (informational, operations to be carried out, list of services of the object or others), either directly from the object itself or through the system, from organizations or users that allow it to be carried out;
(ix) send data or request data related to an object to external data sources (such as manufacturers or regulatory bodies, among others); and
(x) receive data related to an object from an external data source.

8. The procedure according to claim 1, wherein a spatially locatable object records information in one or more databases (internal and/or external), as information on the location of the object and associated objects, among others, such registered data being included within one or more types of data such as public, private, regulatory and/or emergency data, among others, for use by both the object itself and other objects or entities related to objects and with permissions to access that data.

9. The procedure according to claim 1, wherein it also includes:
(xi) automatically detect the presence of a spatially locatable object, within a radius of action (by detecting in the environment by the object or by detecting nearby objects performed in the system);
(xii) send a notification to an object and to a user, owner or organization related to it, indicating the reason and urgency;
(xiii) verify that the data related to a particular object are real, for example by checking it into a reliable data source such as a regulatory body or by verifying the authenticity of such data in a DLT or blockchain; and
(xiv) establish a connection to the object once the authenticity of the object data has been verified.

10. The procedure according to claim 1, wherein data and instructions is sent and received to or from the object, carried out by means of the appropriate security measures in each specific case, to carry out certain operations by means of the services available for the object specified in the DID document of the object or in the system (without ruling out other possible specifications such as in an organization or in a regulatory body).

11. The procedure according to claim 1, wherein an individual is identified and relevant data from the identifier of a spatially locatable object is obtained if the DID document associated with it or an external data source referenced by the DID document contains an individual identifier or a reference to personal data that allows such identification, such as a universal donor identifier that allows access to necessary information such as blood group and other relevant health data in case of emergencies and natural disasters.

12. The procedure according to claim 1, wherein a spatially locatable object sends information to both an emergency/civil protection service and to another spatially locatable object, such as a robot or smart device whose owner is an emergency/civil protection service, without discarding others, either automatically at the time of a certain event or manually by the intervention of the user and/or its owner or legal guardian, including their own identifier, data provided manually by the user, data previously programmed to be shared and sent in certain situations and, optionally, identification and personal data of the user, owner and/or legal guardian.

13. The procedure according to claim 1, wherein from a service of a spatially locatable object, implemented for example by means of a REST API, it identifies all the spatially locatable objects related to it from their respective identifiers, as well as establish communication and send notifications to all or to some of them.

14. The procedure according to claim 1, wherein both an authorized human user through an application and a computer system, an authorized service, an authorized spatially locatable object such as a smart device, a connected machine, among others, sends data and instructions to one or more spatially locatable objects to establish actions and coordinate operations.

15. The procedure according to claim 1, wherein a spatially locatable object may contain information such as a list of objects and individuals that have been authorized and/or have accessed said object or its services, accesses made to the different services and/or to different types of information of the object (such as public, private, emergencies, regulatory information), date and time of those accesses, among other possible information; the authentication can be made through multifactor authentication and the multifactor authentication can be made by reading a PIN code, QR or NFC codes and/or biometric data among others, being the multifactor authentication first done in a concrete spatially locatable object and then in a spatially locatable object of an individual which can be also related to the first spatially locatable object; for example, the access to a spatially locatable object such as a certain restricted area in a hospital can be made through both a peripheral security device which is part of that restricted area (child object of that spatially locatable object) and a pre-authorized smartphone of an individual in that spatially locatable object (also considered as a child object of the first spatially locatable object).

16. The procedure according to claim 1, wherein a spatially localizable object may contain information on the processes carried out and different information on these processes such as who made a request to the object, who authorized it (if approved was required), participants, tasks performed and results, products obtained or manufactured (if applicable), among other possible information; These processes are referenced to information inside or outside the DID document as public, private, emergency, and/or regulatory information, among others, and can be linked to different identifiers such as medical process identifier, biological product identifier, without ruling out others.
